(19) United States
(12) Patent Application Publication
(10) Pub. No.: US 2023/0226330 A1
(43) Pub. Date: Jul. 20, 2023

(54) VASCULAR ACCESS DILATOR APPARATUS AND METHOD OF USE

(71) Applicant: D & J SOLUTIONS, LLC, Philadelphia, PA (US)

(72) Inventors: GERALD WALTER BRONIEC, Philadelphia, PA (US); DAVID HAMMILL, Philadelphia, PA (US)

(73) Assignee: D & J SOLUTIONS, LLC, Philadelphia, PA (US)

(21) Appl. No.: 18/084,713

(22) Filed: Dec. 20, 2022

Related U.S. Application Data
(62) Division of application No. 16/782,942, filed on Feb. 5, 2020.
(60) Provisional application No. 62/801,377, filed on Feb. 5, 2019.

(51) Int. Cl. A61M 29/00; A61M 25/06; A61B 17/3209; A61B 17/34

(52) U.S. Cl. CPC A61M 29/00; A61M 25/0606; A61B 17/32093; A61B 17/34; A61M 2210/12; A61B 2090/061

(57) ABSTRACT

An improved vascular access dilator apparatus consisting of a wire, a blade with a locking safety mechanism, and straight but malleable dilator with a smooth surface having a lumen just large enough for the wire to slide easily in and out. The safety sheath covers the blade once an incision in the dermis is made, thereby protecting both the patient and caregiver.

### CROSS REFERENCE TO RELATED APPLICATION

The present application is a division of pending U.S. patent application Ser. No. 16/782,942 filed on Feb. 5, 2020, that claimed the benefit of U.S. Provisional Application No. 62/801,377 filed on Feb. 5, 2019. Said U.S. patent application Ser. No. 16/782,942 is incorporated by reference herein. As a division thereof, this application claims the benefit of U.S. Provisional Application No. 62/801,377 filed on Feb. 5, 2019.

### FIELD OF THE INVENTION

The present invention relates to a vascular access dilator apparatus for medical use and the method of use thereof.

### BACKGROUND

Vascular access of a peripheral venous or arterial insertion site via Seldinger technique can be challenging and dangerous for both the patient and caregiver. However it is a requirement for the insertion of a flexible, sterile thin plastic tube known as catheters as treatment devices. Most of all it enables an effective method of drawing blood or delivering medications, blood products, or nutrition into a patient's bloodstream.

This access can also be useful for the placement of cannulas such as arterial and/or venous which can remove deoxygenated venous blood and deliver oxygenated arterial blood. In the world of medical devices there are quite a few options for access but unfortunately none offer a complete apparatus that encompasses all the parts necessary for safe and effective access.

Although it is a fairly straightforward procedure, factors such as body habitus, peripheral artery disease, and ongoing cardiac arrest all complicate its achieval. After vascular access is achieved, a small incision in the dermis of the patient's skin is performed so that the site can accommodate the cannula and the series of dilators that facilitate the cannula's insertion via stepwise dilation.

During cannula insertion Seldinger technique is utilized for vascular access. This is followed by small incision in the dermis and then a series of dilations in a stepwise manner allows the artery or vein to accommodate the cannula that will be placed. This device allows for a quick and simple process by the use of a 1-time dilator that has a small blade integrated into the system. Instead of serial dilation and reaching for multiple instruments which requires more time and tools.

An example of a dilator for the access of a peripheral venous or arterial insertion can be seen in U.S. Patent Application number US 2015/0297875A1, which describes the device and method capable of gaining peripheral access. This device requires multiple steps and pieces which in the setting of needing access means a crowded and messy field, along with multiple steps and processes. In an emergency setting the whole scenario becomes exponentially more complicated, dangerous, unsafe for patients along with caregivers, and much more cumbersome with the additional responsibility of grim outcome for the patient.

Therefore a need for a novel vascular dilator apparatus exist. There is also a need for a complete apparatus that already includes within its content the pieces needed for safe and effective access.

### SUMMARY OF THE INVENTION

The present invention comprises a novel vascular dilator apparatus consisting of the following a 0.35″ wire, a Blade with a locking safety mechanism, a dilator that is 7 cm long, straight but malleable and with smooth surface. The dilator has a lumen just large enough for the wire to slide easily in and out. The safety sheath is intended to cover the blade once an incision in the dermis is made. This protects both the patient and caregiver. This apparatus is intended to enter the body through the fascia and vessel wall therefore dilating the region up to a prescribed size.

### BRIEF DESCRIPTION OF THE DRAWINGS

Some embodiments of the present invention are illustrated as an example and not limited by the figures of the accompanying drawings, in which like references may indicate similar elements and in which:

FIG. 1 depicts a side perspective view of one example of a vascular access dilator apparatus.

FIG. 2 depicts a close up perspective view of the blade, safety device, inserted wire and french sizes used for sizing.

FIG. 3 depicts a top view of the vascular access dilator apparatus.

FIG. 4 depicts a underside view of the vascular access dilator apparatus.

FIG. 5 depicts a close up view of the blade safety device along with the french sizes and the wire that is inserted through the device.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an, ” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Vascular access using this dilator apparatus are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

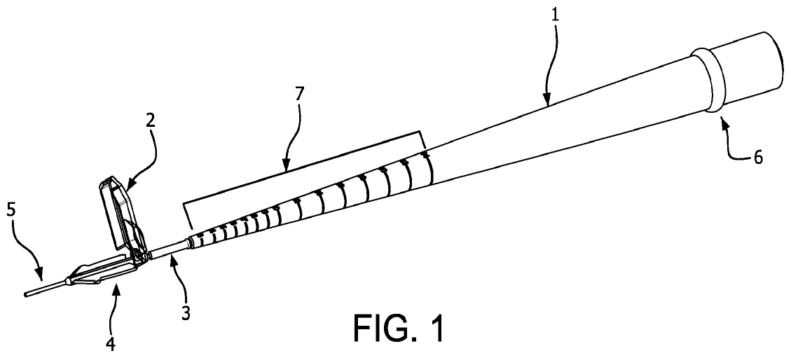

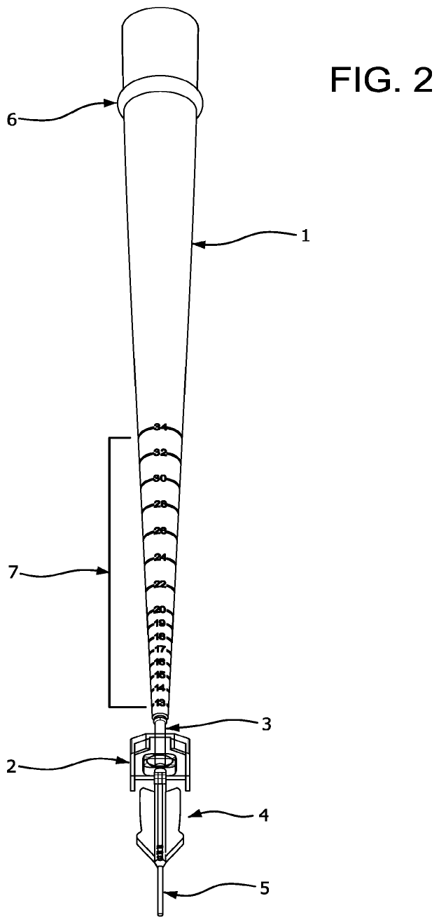

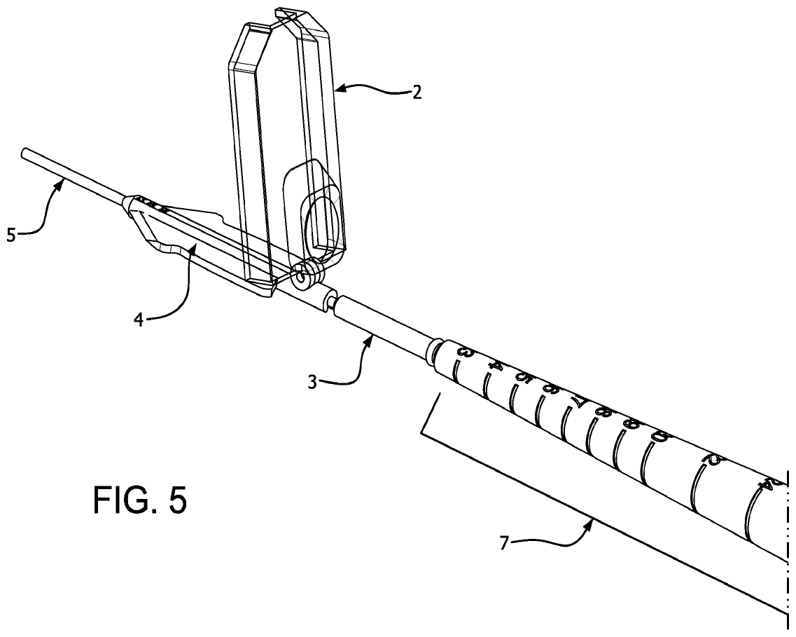

A 21 gauge needle is inserted into a patient's vessel. This hollow needle creates a passageway into the targeted vessel, referred to as “access.” FIG. 1 depicts wire 5, which is then inserted through the needle into that patient's bloodstream, either an artery or vein. Once the wire is satisfactorily placed into the lumen of the vessel, the needle is slid over the wire, away from the patient, and off the wire. FIG. 2 shows blade 4, which slides down the wire to make a small incision in the patient's dermis to facilitate dilation. Additionally, FIGS. 2 and 5 depict markings that correlate with french sizes 8 for vessel cannula insertion. After the incision is made, the blade is withdrawn from the patient and covered by safety cover 2 as seen in FIG. 2. Lastly, the covered blade is removed from the wire and disposed per facility procedure.

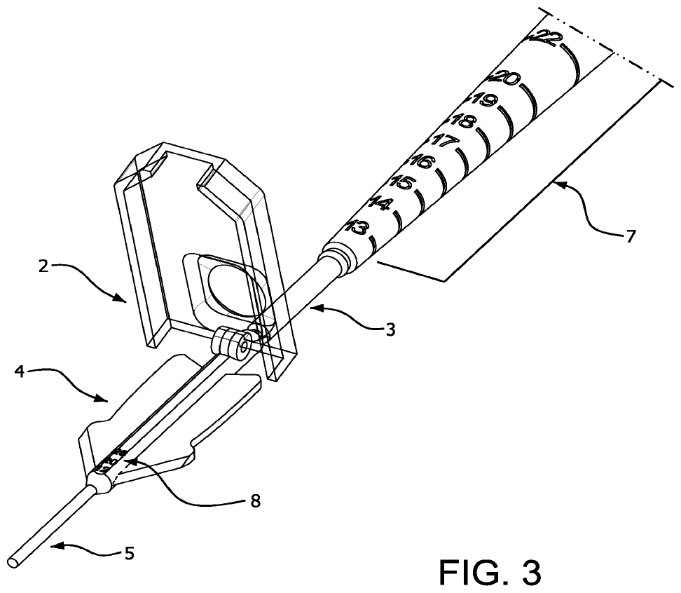

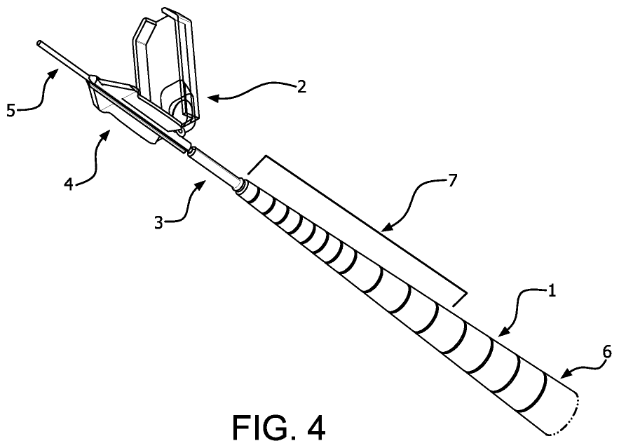

Dilator 1 then slides down wire and is inserted into the lumen of the patient's vessel to the desired french size as shown in FIGS. 3 and 4. Sizes shown in FIG. 3 refer to the depth that the dilator can be inserted into patient to achieve desired dilation size. French sizes 7 are printed on the side of the cannula with circumferential markings so the user can properly dilate. Additionally, band 6 can be placed onto the prescribed size on dilator 1 to prevent over dilation.

Once dilation with apparatus is complete the dilator is removed from the wire and taken removed. Desired cannula is placed along wire, inserted into patient's vessel, and wire is removed.

What is claimed is:

1. A method for introduction of a treatment device into a body vessel of a patient and safe disposal of apparatus therefor comprising the steps of:
inserting a hollow needle into said vessel;
inserting a wire longitudinally into said needle;
retracting said needle;
sliding a blade on said wire in the direction of said vessel;
using said blade to make an incision in the dermis surrounding said vessel;
withdrawing said blade;
covering said blade with a safety cover retractable therefrom;
disposing of said covered blade;
sliding a cannula on said wire in the direction of said vessel; and
inserting said cannula into said vessel to the depth of the proper French size printed thereon relative to the dermis of said patient,
whereby said cannula is placed in said vessel in a safe manner for proper dilation for introduction of a treatment device.

2. A method for introduction of a treatment device into a body vessel of a patient and safe disposal of apparatus therefor comprising the steps of:
inserting a hollow needle into said vessel;
inserting a wire longitudinally into said needle;
retracting said needle;
sliding a blade on said wire in the direction of said vessel;
using said blade to make an incision in the dermis surrounding said vessel;
withdrawing said blade;
covering said blade with a safety cover retractable therefrom;
disposing of said covered blade;
sliding a cannula on said wire in the direction of said vessel;
placing a band at a prescribed French size printed on said cannula; and
inserting said cannula into said vessel to the depth of the placement of said band relative to the dermis of said patient,
whereby said cannula is placed in said vessel in a safe manner for proper dilation for introduction of a treatment device.
